(19) United States
(12) Patent Application Publication
(10) Pub. No.: US 2018/0016231 A9
(43) Pub. Date: Jan. 18, 2018

(54) METHOD FOR PREPARING STRUCTURED DIRECTING AGENT

(71) Applicant: Johnson Matthey Public Limited Company, London (GB)

(72) Inventors: Steven COLLIER, Devens, MA (US); Da-Ming GOU, Burlington, MA (US); Jeremy WILT, Devens, MA (US)

(21) Appl. No.: 15/133,811

(22) Filed: Apr. 20, 2016

Related U.S. Application Data
(60) Provisional application No. 62/150,015, filed on Apr. 20, 2015.

(51) Int. Cl. C07D 211/14; C07C 209/20; C01B 39/48

(52) U.S. Cl. CPC C07D 211/14; C07C 209/20; C01B 39/48

(57) ABSTRACT

Provided is a method for preparing a structure directing agent (SDA) for crystalline molecular sieve synthesis comprising the steps of (a) hydrolyzing analkyl sulfate counterion of a quaternary ammonium salt to produce an organic ammonium salt having a hydrogen sulfate counterion; and (b) contacting the organic ammonium salt having the hydrogen sulfate counterion with a source of hydroxide in solution to form an organic ammonium salt having a hydroxide counterion; wherein the organic ammonium salt is a structure directing agent (SDA) for crystalline molecular sieve synthesis.

### BACKGROUND

1. Field of Invention

The present invention relates to a method for preparing an organic structure directing agent that is useful in zeolite synthesis.

2. Description of Related Art

Zeolites are porous crystalline or quasi-crystalline structures having a framework constructed of inorganic oxides, such as silicates and aluminates, which are arranged in a regular repeating pattern. These frameworks consist of patterns of cages and channels which give rise to the molecularly porous nature of the zeolite. Each unique zeolite framework recognized by the International Zeolite Association (IZA) Structure Commission is assigned a three-letter code to designate the framework type, such as CHA (chabazite), BEA (beta), and MOR (mordenite).

Certain zeolite crystals can be formed by mixing various oxides in the presence of an organic structure directing agent (SDA), such as quaternary organic tetramethylammonium (TMA) salts. The SDA serves as a template of sorts around which various building units of a zeolite can develop and join together to produce the crystalline lattice structure. Once the zeolite crystals are formed, they can be separated from their host mother liquor and dried. The resulting crystals are then typically heated in order to thermally decompose the interior SDA molecules, at which point the SDA remnants can be extracted from the zeolite crystal, thus leaving only the porous oxide zeolite framework.

SDA are often complex molecules which require time-consuming and multi-step processes to synthesize. The relatively high cost of SDAs and the fact that they are consumed during zeolite synthesis are significant contributors to the cost of manufacturing a zeolite. Accordingly, there remains a need in the art for more efficient, cost effective synthetic routes for manufacturing SDAs on commercial scale. This invention satisfies this need amongst others.

### SUMMARY OF THE INVENTION

It has been found that a cyclic amine can be quickly and easily converted into a functional SDA, such as 1,1,3,5-tetramethylpiperidin-1-ium hydroxide. For example, reacting a cyclic amine with an alkyl sulfate produces a novel intermediate quaternary ammonium salt having an alkyl sulfate counter ion. Reacting this quaternary ammonium/alkyl sulfate salt with a hydrolysis agent, such as sulfuric acid, converts the alkyl sulfate counter ion into a hydrogen sulfate counter ion, which can then be reacted with a hydroxide source to create the hydroxide form of the SDA and an inorganic sulfate salt, the latter of which forms a precipitate which can be easily removed from the solution. Thus, the present process described herein is a simple, yet novel route for the synthesis of an SDA. Advantageously, the present method also directly produces the SDA in hydroxide form which can be more readily used in zeolite synthesis. In addition, the present invention can yield an SDA that having low concentrations of alkali metals and sulfur.

Accordingly, provided is a method for preparing a structure directing agent (SDA) for crystalline molecular sieve synthesis comprising the steps of (a) hydrolyzing an alkyl sulfate counterion of a quaternary ammonium salt to produce a quaternary ammonium salt having a hydrogen sulfate counterion; and (b) contacting the quaternary ammonium salt having the hydrogen sulfate counterion with a source of hydroxide in solution to form a quaternary ammonium salt having a hydroxide counterion; wherein the quaternary ammonium salt is a structure directing agent (SDA) for crystalline molecular sieve synthesis.

In another aspect, provided is a novel composition comprising at least one of N,N-Dimethyl-3,5-dimethylpiperidinium methyl sulfate; N,N-Diethyl-2,6-dimethylpiperidinium methyl sulfate; N,N-Dimethyl-9-azoniabicyclo[3.3.1]nonane methyl sulfate; N,N-Dimethyl-2,6-dimethylpiperidinium methyl sulfate; N-Ethyl-N-methyl-2,6-dimethylpiperidinium alkyl sulfate; N,N-Diethyl-2-ethylpiperidinium ethyl sulfate; N,N-Dimethyl-2-ethylpiperidinium methyl sulfate; N-Ethyl-N-methyl-2-ethylpiperidinium alkyl sulfate; N-Ethyl-N-propyl-2,6-dimethylpiperidinium alkyl sulfate; and 2,2,4,6,6-Pentamethyl-2-azoniabicyclo[3.2.1] octane methyl sulfate.

In another aspect, provided is a composition comprising at least one of N,N-Dimethyl-3,5-dimethylpiperidinium hydrogen sulfate; N,N-Diethyl-2,6-dimethylpiperidinium hydrogen sulfate; N,N-Dimethyl-9-azoniabicyclo[3.3.1]nonane hydrogen sulfate; N,N-Dimethyl-2,6-dimethylpiperidinium hydrogen sulfate; N-Ethyl-N-methyl-2,6-dimethylpiperidinium hydrogen sulfate; N,N-Diethyl-2-ethylpiperidinium hydrogen sulfate; N,N-Dimethyl-2-ethylpiperidinium hydrogen sulfate; N-Ethyl-N-methyl-2-ethylpiperidinium hydrogen sulfate; N-Ethyl-N-propyl-2,6-dimethylpiperidinium hydrogen sulfate; and 2,2,4,6,6-Pentamethyl-2-azoniabicyclo[3.2.1] octane hydrogen sulfate.

In another aspect, provided is a method for preparing a structure directing agent (SDA) for crystalline molecular sieve synthesis comprising the steps of: (a) reacting a quaternary ammonium-SDA precursor with one or more dialkylsulfates in solution to form a first intermediate solution; (b) contacting the first intermediate solution with an acid or base to produce a second intermediate solution containing a hydrogen sulfate anion; and (c) contacting the second intermediate solution with a base to produce a final solution comprising an hydroxide form of an ammonium-based SDA.

In yet another aspect of the invention, provided is a method for preparing a structure directing agent (SDA) for crystalline molecular sieve synthesis comprising the steps of: (a) reacting an optionally substituted pyridine-based SDA precursor with one or more dialkylsulfates in solution to form a first intermediate solution containing a pyridinium alkyl sulfate; (b) reducing the first intermediate solution of pyridinium alkyl sulfate to provide a second intermediate solution of piperidinium alkyl sulfate; (c) reacting the second intermediate solution of piperidinium alkyl sulfate with one or more dialkylsulfates in solution to form a third intermediate solution of piperidinium alkyl sulfate; (d) contacting the third intermediate solution with an acid or base to produce a fourth intermediate solution of piperidinium hydrogen sulfate; and (e) contacting the fourth intermediate solution with a base to produce a final solution comprising an hydroxide form of an ammonium-based SDA.

### BRIEF DESCRIPTION OF THE DRAWINGS

FIG. 1 is a diagram showing synthesis of an SDA according to an embodiment of the invention; and

FIG. 2 is a diagram showing synthesis of N,N-dimethyl-3,5-dimethylpiperidinium hydroxide according to an embodiment of the invention.

### DETAILED DESCRIPTION OF PREFERRED EMBODIMENTS OF THE INVENTION

Provided are improved methods for preparing structure directing agents (SDAs) useful for crystalline molecular sieve (e.g., zeolite) synthesis. In certain embodiments, the methods are improved, in part, by rapid formation of the desired SDA in hydroxide form, without the need for a metal ion exchange process (e.g., by ion exchange resin). SDAs produced by the present method include those useful for the synthesis of zeolites having one or more of the following frameworks: CHA, AEI, AFX, AFT, ERI, and LEV, including intergrowths of two or more of these. Such SDAs include N,N-Dimethyl-3,5-dimethylpiperidinium hydroxide and N,N-Dimethyl-2,6-diethylpiperidinium hydroxide.

In certain embodiments, the SDA synthesis involves the conversion of the quaternary ammonium salt counter ion from an alkyl sulfate to a hydrogen sulfate and then to a hydroxide by a process wherein the sulfate moiety forms a solid precipitate which can be easily removed from the system. Preferably, the method involves the steps of: (a) hydrolyzing an alkyl sulfate counterion of a quaternary ammonium salt to produce a quaternary ammonium salt having a hydrogen sulfate counterion; and (b) contacting the organic ammonium salt having the hydrogen sulfate counterion with a source of hydroxide in solution to form an organic ammonium salt having a hydroxide counterion; wherein the hydroxide form of the organic quaternary ammonium salt is useful as a structure directing agent (SDA) for crystalline molecular sieve synthesis. Step (a) can occur in the presence of an acid, such as sulfuric acid, or a hydroxide. Preferably, the source of hydroxide in step (b) is an alkali metal hydroxide or ammonium hydroxide. In certain embodiments, the methods further involve the step of extracting the SDA from the reaction solution. In certain embodiments, the methods further involve formation of a quaternary ammonium salt precursor by quaternizing a cyclic amine using an alkyl sulfate. In certain embodiments, the methods further involve the step of alkylating a starting material, such as 3,5-lutidine or 3,5-dimethylpiperidine followed by reduction to produce the cyclic amine, preferably using the same type of alkyl sulfate that is used in step (a). Turning to step (a) of the process, useful quaternary ammonium salts preferably include a non-aromatic, 5-, or 6-membered cyclic ammonium ion, wherein the nitrogen is bonded to two additional alkyls or forms the spirocyclic center second ring structure. In certain embodiments, the quaternary ammonium contains the following moiety:

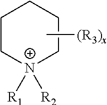

wherein R₁ and R₂ are independently an alkyl or members of a ring structure, X is an integer from 1 to 5 and each R₃ is independently an alkyl functional group. As used herein, the term “alkyl” encompasses straight chained and branched C₁-C₅-alkyl or cycloalkyl groups.

Preferred ammonium ions are 6-membered monocyclic rings having two alkyl groups at the N-position. Examples of alkyl groups are, in particular, methyl, ethyl, propyl, isopropyl, n-butyl, sec-butyl, isobutyl, tert-butyl, n-pentyl, 2-pentyl, 2-methyl butyl, 3-methyl butyl, 1,2-dimethyl propyl, 1,1-dimethylpropyl, and 2,2-dimethylpropyl. Particularly preferred alkyl groups at the N-position of the ammonium ion include methyl, ethyl, and n-propyl. The two alkyl groups at the N-position can be the same, such as dimethyl or diethyl, or can be different, such as ethyl and methyl or n-propyl and ethyl.

One or more additional alkyl substitutions can be made at other locations on the ring structure. For example, alkyl functional groups can be substituted at the -2-, -3-, -4-, -5-, and/or -6-positions. The number of additional substitutions is preferably one or two. Where two additional substitutions are made, they are preferably symmetric with respect to the N-atom on the ammonium ring. Preferably, alkyl substitutions are made at the -2,6- or the -3,5-positions. The two additional alkyl groups can be the same, such as dimethyl, or diethyl or can be different, such as ethyl-methyl, n-propyl-ethyl. Particularly preferred are dimethyl substitutions and diethyl substitutions.

Specific examples of preferred quaternary ammonium salts are those having an ion selected from the group consisting of N,N-Dimethyl-3,5-dimethylpiperidinium; N,N-Diethyl-2,6-dimethylpiperidinium; N,N-Dimethyl-9-azoniabicyclo[3.3.1]nonane; N,N-Dimethyl-2,6-dimethylpiperidinium; N-Ethyl-N-methyl-2,6-dimethylpiperidinium; N,N-Diethyl-2-ethylpiperidinium; N,N-Dimethyl-2-ethylpiperidinium; N-Ethyl-N-methyl-2-ethylpiperidinium; N-Ethyl-N-propyl-2,6-dimethylpiperidinium; and 2,2,4,6,6-Pentamethyl-2-azoniabicyclo[3.2.1] octane, with N,N-Dimethyl-3,5-dimethylpiperidinium being particularly preferred.

Preferably, the intermediate quaternary ammonium salts produced via this process also comprise an alkyl sulfate counterion. Particularly preferred alkyl sulfate counterions include methyl sulfate and ethyl sulfate. Accordingly, the present invention encompasses novel salts such as 1,1,3,5-tetramethylpiperidin-1-ium methyl sulfate. The salt is preferably in the form of an aqueous solution.

Step (a) preferable involves hydrolyzing the quaternary ammonium/alkyl sulfate salt to form a quaternary ammonium salt having a hydrogen sulfate counterion. This hydrolysis can be achieved, for example, by contacting the quaternary ammonium/alkyl sulfate salt with sulfuric acid or a hydroxide. Useful hydroxides include alkali metal hydroxides and ammonium hydroxides. Examples of alkali metal hydroxides include lithium hydroxide, sodium hydroxide, and potassium hydroxide. In certain embodiments, the sulfuric acid or source of hydroxide is added directly to a solution of the quaternary ammonium/alkyl sulfate salt to form a reactive admixture. Typically, the hydrolysis reaction will proceed from about 30 minutes to several hours at a temperature of about 25-125° C. The alkyl-alcohol byproduct, such as methanol, of the hydrolysis reaction can be removed from the aqueous solution by azeotropic distillation to achieve an overall conversion of alkyl sulfate to hydrogen sulfate of above 95%, and preferably above 99%. Preferably, step (a) is performed using a non-organic solvent.

Step (b) involves contacting the solution of organic quaternary ammonium/hydrogen sulfate salt with a source of hydroxide to form an organic ammonium salt having a hydroxide counterion. Preferred sources of hydroxide include alkali metal hydroxides, such as lithium hydroxide, sodium hydroxide, and potassium hydroxide. In certain embodiments, step (b) results in a SDA having little or no alkali metal content, despite the use of alkali metal during synthesis. Preferably, the alkali content of such SDAs is less than 5 weight percent based on the total weight of the SDA, more preferably less than 3 weight percent, and even more preferably less than 1 weight percent.

Step (b) can be performed using an organic solvent, such as isopropyl alcohol, or a non-organic solvent. Preferably, the alkali hydroxide and the organic ammonium salt/hydrogen sulfate salt are combined into a system under conditions effective to form an alkali metal sulfate which will precipitate from the solution. The solid inorganic sulfate can then be removed from the solution by any known means, such as filtration. The filtrate contains the organic ammonium salt in hydroxide form. Thus, the methods of the present invention can be used to prepare an SDA in hydroxide form without an ion exchanger such as an ion exchange resin.

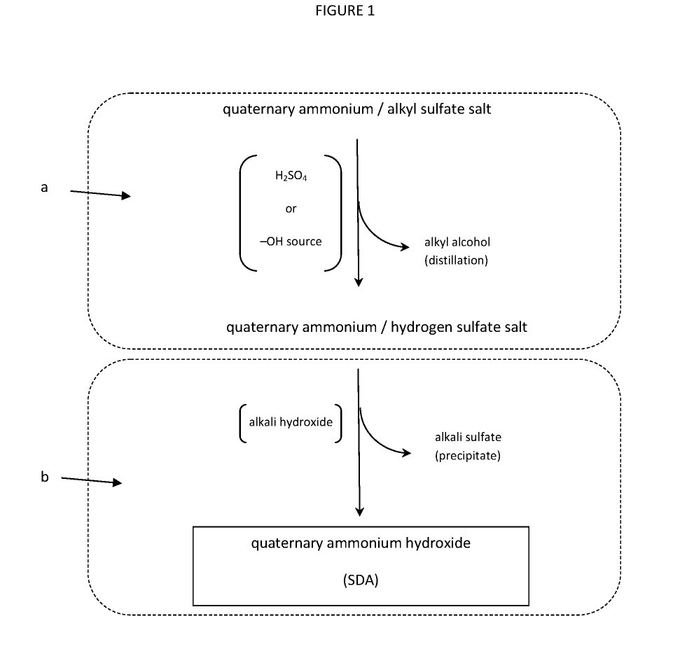

A certain embodiment of the process, including steps (a) and (b), is shown in FIG. 1.

The quaternary ammonium/alkyl sulfate salt of step (a) can be prepared from a nitrogen-containing precursor. Examples of useful precursor compounds include nitrogen containing substituted five-member or six-member ring compounds, such as a substituted piperidine or a substituted pyridine. In certain embodiments, the precursor compounds include methyl, ethyl, and/or propyl substitution(s) at one or two ring positions, preferably the -2-, the -2- and -6-, or the -3- and -5-positions. Examples of such precursors molecules include 3,5-dimethylpiperidine and 3,5-lutidine.

Alkylation of the precursor compound preferably involves the addition of a methyl, ethyl, or propyl group to the compound's -1-position. The alkylating agent is preferably a dialkyl sulfate, such as dimethyl sulfate, diethyl sulfate, methylethyl sulfate, dipropyl sulfate, methylpropyl sulfate, ethylpropyl sulfate, and mixtures thereof, with dimethyl sulfate being particularly preferred. In required, the alkylated precursor compound can be further treated to yield a corresponding non-aromatic ring. Examples of preferred methylations include:

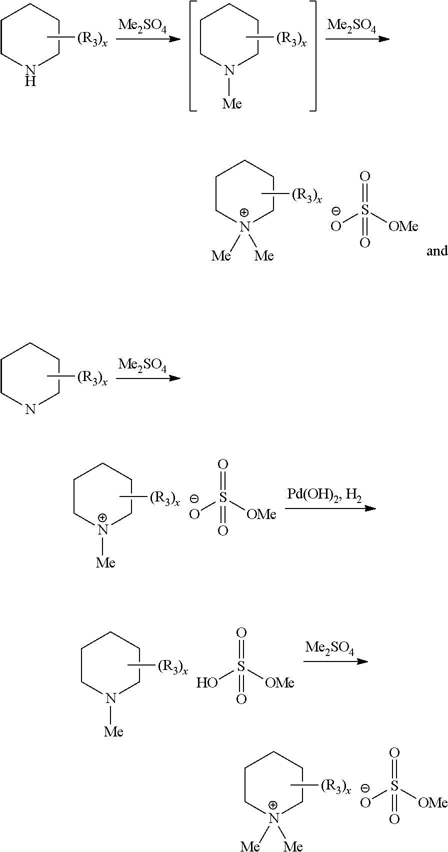

where x=an integer from 0 to 5, preferably 1 or 2;

Alkylation of the precursor compound yields a substituted cyclic amine having an alkyl group on at the -1-position. Examples of substituted cyclic amines useful in the present invention include alkyl substituted N-methylpiperidine, alkyl substituted N-ethylpiperidine, and alkyl substituted N-propylpiperidine. The alkyl substitutions for these compounds include methyl, ethyl, propyl, and/or butyl at the -2-, -3-, -5-, and/or -6-positions. In addition to the alkyl substitution at the -1-position, these compounds can have one, two, or three additional substitutions. In certain embodiments, methyl substitutions are made at -2- and -6-positions or at the -3- and -5-positions. In certain embodiments, an ethyl substitution is made at the -2-position. For example, preferred substituted piperidines have two or three alkyl substitutions such as trimethylpiperidine, triethylpiperidine, dimethylethylpiperidine, and methylethylpiperidine, particularly those having a methyl or ethyl substitution at the -1-position. Other substitutions preferably include methyls substituted at the -2- and -6-position, methyls substituted at the -3- and -5-positions, or an ethyl substituted at the -2-position. A particularly preferred substituted piperidine is 1,3,5-trimethyl piperidine.

In certain embodiments, a tertiary cyclic amine precursor is quaternized with an alkyl sulfate to yield a quaternary ammonium salt. Useful alkyl sulfates for the quaternization include those described above. In certain embodiments, the same type of alkyl sulfate used for alkylation of the precursor compound can be used for the quaternization. For example, dimethyl sulfate can be used both for alkylation of the precursor compound and for quaternization of the corresponding tertiary cyclic amine. In other embodiments, different alkyl sulfates can be used for alkylation of the precursor compound and for the quaternization of the corresponding tertiary cyclic amine.

The quaternization preferably involves an alkyl or ring substitution at the -1-position. Preferred substitutions yield a -1,1-dimethyl, -1,1-methylethyl, or -1,1-diethyl moiety.

### EXAMPLES

### Example 1

### Synthesis of 1,3,5-trimethylpiperidinium methyl sulfate from 3,5-lutidine

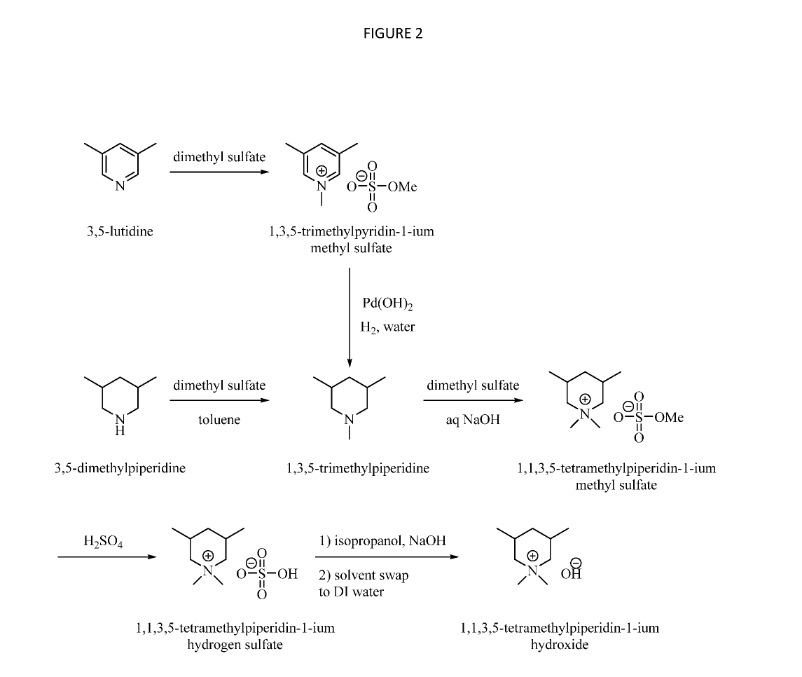

Referring to FIG. 2, 3,5-lutidine (10.47 g) was placed in a flask and stirred at −10° C. Dimethyl sulfate (12.89 g) was added in portions, keeping the batch temperature below 60° C. The batch was then stirred at 40-50 C until the reaction was complete. Once complete, the batch was diluted with water to prepare an approximately 60 wt % solution of 1,3,5-trimethylpyridin-1-ium methyl sulfate.

This solution was added to a stainless steel reaction vessel, and sponge nickel catalyst (Alfa Aesar) was added to the batch. Hydrogen (16 bar) was then introduced at 25° C., and the batch was stirred for 5 hours. Upon completion, the catalyst was filtered to provide 1,3,5-trimethylpiperidinium methyl sulfate (99%) as a solution in water.

### Example 2

### Synthesis of N,N-Dimethyl-3,5-dimethylpiperidinium hydroxide

Again Referring to FIG. 2, to a clean and dry jacketed reactor was charged 3,5-Dimethylpiperidine (11 kg) and Toluene (11 L). The mixture was cooled to 0-10° C. Dimethyl sulfate (12.2 kg) was charged carefully while maintaining the batch temperature <70° C. After the addition was complete, the batch was cooled to 10-25° C. and stirred for at least 1 h.

The batch was then further cooled to 5-10° C. Dimethyl sulfate (13.5 kg) was added to the reaction mixture followed by purified water (11.0 kg). A solution of sodium hydroxide (10.8 kg water; 4.6 kg sodium hydroxide) was added at 5-10° C., maintaining the batch temperature below 10° C.

Once the reaction was complete, a solution of sulfuric acid (4.7 kg) in purified water (4.8 kg) was added to the batch over 1 hour, maintaining the batch temperature <50° C. The batch was heated to 95-100° C. and solvent was removed by distillation until approximately 2 volumes of solvent with respect to the starting material was collected.

Purified water (2-3 L) was then added to the batch and the distillation was continued until approximately 2-3 L solvent was removed. This water chase was repeated twice. Finally, the batch was concentrated by distillation until approximately 38-40 L remained. The batch was cooled to approximately 25° C., then isopropanol (25.9 kg) was added to the batch.

The batch was cooled to 10° C., then a solution of sodium hydroxide (16.2 kg) and purified water (16.2 kg) was added to the batch while maintaining the temperature <46° C. Additional isopropanol (5.5 kg) was charged to the resulting slurry. The batch was cooled to 0-5° C. and filtered. The solids were washed with isopropanol (3×5.5 kg). The resulting filtrate was evaporated at <45° C., and residual isopropanol was driven off with water to provide N,N-Dimethyl-3,5-dimethylpiperidinium hydroxide as a 50 wt % solution in water (97% yield).

### Example 3

### Synthesis of AEI zeolite (SAR=22)

A reaction gel of (molar) composition of 60 parts SiO₂, 1.2 parts Al₂O₃, 13.41 parts Na₂O, 9.5 parts N,N-diethyl-2,6-dimethylpiperidinium hydroxide (22.23 wt % solution), and 2721 parts H₂O was prepared as follows: About 130.6 grams of a source of silica (30 wt % SiO₂) was changed into a 1 Liter stainless steel autoclave with the agitator set to rotate at 300 rpm. About 341.4 g of 1N NaOH was mixed in a beaker with 98.3 g of the template. About 7.6 g of ammonium exchanged Y zeolite was added to this mixture. The mixture was stirred at room temperature for 10-15 min before being added to the colloidal silica in the autoclave. The autoclave was sealed and mixing continued, at room temperature, for a further 10 min before being heated to 135° C. The temperature was maintained for 12 days then the autoclave was cooled to room temperature, the product discharged then filtered, washed with demineralized water and dried at 110° C. overnight.

The resulting product was analyzed by X-ray powder diffraction and found to be a highly crystalline AEI type zeolite.

What is claimed is:

1. A method for preparing a structure directing agent (SDA) for crystalline molecular sieve synthesis comprising the steps of:
a. hydrolyzing analkyl sulfate counterion of a quaternary ammonium salt to produce an organic ammonium salt having a hydrogen sulfate counterion; and
b. contacting the organic ammonium salt having the hydrogen sulfate counterion with a source of hydroxide in solution to form an organic ammonium salt having a hydroxide counterion; wherein the organic ammonium salt is a structure directing agent (SDA) for crystalline molecular sieve synthesis.

2. The method of claim 1, wherein the contacting step further comprises precipitating a sulfate from the solution.

3. The method of claim 2, wherein the precipitated sulfate is removed from the solution by filtration.

4. The method of claim 1, wherein the hydrolyzing step comprises contacting the alkyl sulfate counterion of an organic ammonium salt with sulfuric acid or hydroxide.

5. The method of claim 1, wherein the source of hydroxide is an alkali metal hydroxide or an ammonium hydroxide.

6. The method of claim 1, wherein the organic ammonium salt is a structure directing agent (SDA) for synthesis of a molecular sieve having a framework selected from CHA, AEI, AFX, ERI, LEV, AFT, or an intergrowth of two or more of these.

7. The method of claim 1, wherein the organic ammonium salt is a structure directing agent (SDA) for synthesis of a molecular sieve having an AEI framework.

8. The method of claim 1, wherein the organic ammonium salt comprises an ammonium ion selected from N,N-Dimethyl-3,5-dimethylpiperidinium; N,N-Diethyl-2,6-dimethylpiperidinium; N,N-Dimethyl-9-azoniabicyclo[3.3.1]nonane; N,N-Dimethyl-2,6-dimethylpiperidinium; N-Ethyl-N-methyl-2,6-dimethylpiperidinium; N,N-Diethyl-2-ethylpiperidinium; N,N-Dimethyl-2-ethylpiperidinium; N-Ethyl-N-methyl-2-ethylpiperidinium; N-Ethyl-N-propyl-2,6-dimethylpiperidinium; and 2,2,4,6,6-Pentamethyl-2-azoniabicyclo[3.2.1] octane.

9. The method of claim 1, further comprising quaternizing a tertiary amine using a dialkyl sulfate to produce the organic ammonium salt having the methyl sulfate counterion.

10. The method of claim 9, wherein the tertiary amine is selected from the group consisting of 1,3,5-trimethylpiperidine; 1-ethyl-2,6-dimethylpiperidine; 1,2,6-trimethylpiperidine; 1-methyl-2-ethylpiperidine; 1-methyl-9-azoniabycyclo[3.3.1]nonane; 1,2-diethylpiperidine; 1-propyl-2,6-dimethylpiperidine; and 1,2,4,6,6-tetramethyl-1-azoniabicyclo[3.2.1]octane.

11. The method of claim 9, wherein the tertiary amine is 1,3,5-trimethylpiperidine.

12. A composition comprising at least one of N,N-Dimethyl-3,5-dimethylpiperidinium hydrogen sulfate; N,N-Diethyl-2,6-dimethylpiperidinium hydrogen sulfate; N,N-Dimethyl-9-azoniabicyclo[3.3.1]nonane hydrogen sulfate; N,N-Dimethyl-2,6-dimethylpiperidinium hydrogen sulfate; N-Ethyl-N-methyl-2,6-dimethylpiperidinium hydrogen sulfate; N,N-Diethyl-2-ethylpiperidinium hydrogen sulfate; N,N-Dimethyl-2-ethylpiperidinium hydrogen sulfate; N-Ethyl-N-methyl-2-ethylpiperidinium hydrogen sulfate; N-Ethyl-N-propyl-2,6-dimethylpiperidinium hydrogen sulfate; and 2,2,4,6,6-Pentamethyl-2-azoniabicyclo[3.2.1] octane hydrogen sulfate.

13. A method for preparing a structure directing agent (SDA) for crystalline molecular sieve synthesis comprising the steps of: 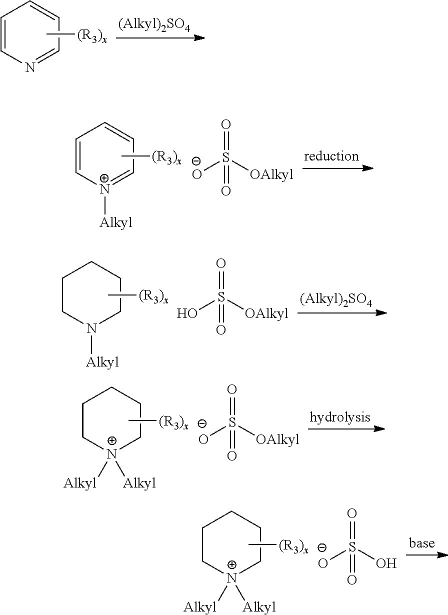 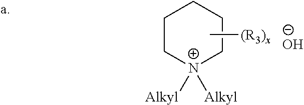
reacting an optionally substituted pyridine-based SDA precursor with one or more dialkylsulfates in solution to form a first intermediate solution containing a pyridinium alkyl sulfate;
b. reducing the first intermediate solution of pyridinium alkyl sulfate to provide a second intermediate solution of piperidinium alkyl sulfate;
c. reacting the second intermediate solution of piperidinium alkyl sulfate with one or more dialkylsulfates in solution to form a third intermediate solution of piperidinium alkyl sulfate;
d. contacting the third intermediate solution with an acid or base to produce a fourth intermediate solution of piperidinium hydrogen sulfate; and
e. contacting the fourth intermediate solution with a base to produce a final solution comprising an hydroxide form of an ammonium-based SDA.
